(19) United States
(12) Patent Application Publication
(10) Pub. No.: US 2018/0369534 A1
(43) Pub. Date: Dec. 27, 2018

(54) RESUSCITATOR

(71) Applicant: SWIRL TECHNOLOGIES PTY LTD, Mitcham (AU)

(72) Inventor: Ian BIRD, Mitcham (AU)

(21) Appl. No.: 15/775,082

(22) PCT Filed: Nov. 10, 2016
(86) PCT No.: PCT/AU2016/051078

§ 371 Date: May 10, 2018

(30) Foreign Application Priority Data
Nov. 10, 2015 (AU) 2015904620

(51) Int. Cl. A61M 16/20; A61M 16/08; A61M 16/04; A61M 16/06; A61M 16/12

(52) U.S. Cl. CPC A61M 16/201; A61M 16/209; A61M 16/0816; A61M 2016/003; A61M 16/06; A61M 16/125; A61M 16/04

(57) ABSTRACT

A resuscitator includes a flow control unit (5). The unit includes: (a) a peak inspirational pressure valve and controller (“PIP valve”) for controlling the pressure of gas delivered to the lungs of a patient, such as an infant, during inhalation so that the pressure does not exceed a selected PIP pressure, (b) a positive end-expiratory pressure valve and flow controller (“PEEP valve”) for allowing gas flow from the lungs through the PEEP valve during exhalation when the pressure in the lungs is above a selected PEEP pressure, and (c) a blow-off pressure relief valve that opens automatically if the gas pressure being delivered to the lungs exceeds a selected safe operation blow-off pressure that is higher than the selected PIP pressure.

### FIELD OF THE INVENTION

The present invention relates to a resuscitator.

The invention relates particularly, although by no means exclusively, to a manually-adjustable resuscitator for infants.

The invention relates particularly, although by no means exclusively, to a manually-adjustable resuscitator that includes a hand-held multiple purpose flow control unit.

### BACKGROUND OF THE INVENTION

Infant resuscitators are important equipment and it is critical that resuscitators be adapted to be used quickly and reliably to deliver required gases, including mixed gases, to infants in respiratory distress.

The gases can be air or oxygen or oxygen-enriched air.

The gases should be delivered to infants at specific pressures which will benefit the resuscitation of the infants while not causing damage to the lungs of the infants.

In this context, it is important that health care professionals be able to monitor and, where necessary, adjust the operating parameters of resuscitators quickly and reliably in often cramped working conditions.

It is also important that resuscitators be safe to use and that there be appropriate safeguards to prevent excess pressure being delivered to infants' lungs which could cause irreparable damage to the lungs.

The above description of the background of the invention is not to be taken as an admission of the common general knowledge in Australia or elsewhere.

### SUMMARY OF THE INVENTION

In broad terms, the invention provides a resuscitator that includes a flow control unit that includes: (a) a peak inspirational pressure valve and controller (“PIP valve”) for controlling the pressure of gas delivered to the lungs of a patient, such as an infant, during inhalation so that the pressure does not exceed a selected PIP pressure, (b) a positive end-expiratory pressure valve and flow controller (“PEEP valve”) for allowing gas flow from the lungs through the PEEP valve during exhalation when the pressure in the lungs is above a selected PEEP pressure, and (c) a blow-off pressure relief valve that opens automatically if the gas pressure being delivered to the lungs exceeds a selected safe operation blow-off pressure that is higher than the selected PIP pressure. The invention provides a resuscitator that includes a flow control unit that, in use, can be positioned at the head end of a patient, such as an infant, and connected to a gas source and to a face mask or endotracheal tube for the patient for supplying air or oxygen or oxygen-enriched air (hereinafter referred to as “gas”) under pressure to the patient, with the flow control unit including: (a) a passage for gas from the gas source to flow through the unit to be delivered to the patient during inhalation and for gas to flow from the patient during exhalation, (b) a peak inspirational pressure valve and controller (“PIP valve”) for controlling the pressure of gas delivered to the lungs via the passage during inhalation so that the pressure does not exceed a selected PIP pressure, (c) a positive end-expiratory pressure valve and flow controller (“PEEP valve”) for allowing gas flow from the lungs via the passage and through the PEEP valve during exhalation when the pressure in the lungs is above a selected PEEP pressure to ensure that there is a minimum pressure (i.e. the selected PEEP pressure) of gas held in the lungs after exhalation, (d) a blow-off pressure relief valve that opens automatically if the gas pressure being delivered to the lungs via the passage exceeds a selected safe operation blow-off pressure that is higher than the selected PIP pressure; and (e) a pressure gauge for measuring the gas pressure in the passage.

The advantages of the resuscitator of the invention over resuscitators known to the applicant include the following advantages:
All of the main flow control components of the resuscitator are in the flow control unit and this unit can be held and operated in one hand and be positioned in an operative position in close proximity of an infant's head.
Being able to position the flow control unit close to the head of an infant provides important operational advantages over other resuscitators known to the applicant.
Providing the hand-held flow control unit with a blow-off pressure relief valve means that any over-pressure of delivery is instantly and automatically released and the healthcare professional can be assured of this by looking at the pressure gauge, which is also conveniently located in the hand-held control unit.
The flow control unit, and all advantageous features of the unit, is close to an infant's head and this makes it possible for much more accurate readings of the infant's airway pressures to be taken and a quicker response to any emergency arising from unforeseen factors.
The flow control unit minimizes dead space.
The flow control unit makes it possible to have direct connection to the gas source via standard tubing and this makes it easier to use the resuscitator.
The gas supply flow meter nipple (described below) allows the connection of either smaller or larger diameter tubing between the gas source and the flow control unit.

In use, the flow control unit of the above-described resuscitator is connected by suitable tubing to a gas source. The gas source may be any suitable gas source, such as a portable gas cylinder or a wall-mounted gas flow outlet in a hospital room. In a calibration stage using a test lung or other suitable test device, a healthcare professional adjusts the PIP and PEEP controllers separately to required selected operating PIP and PEEP pressures for the PIP and PEEP valves. The healthcare professional then connects the resuscitator to a face mark or an endotracheal tube for a patient, such as an infant, and commences resuscitation of the patient. More particularly, the healthcare professional selectively closes and opens the PEEP valve to a required inhalation and exhalation rhythm for the patient. When the PEEP valve is closed by the healthcare professional positioning a thumb or finger over the valve, gas flows from the gas source through the flow control unit to the lungs of the patient at a pressure up to the selected PIP pressure. The PIP valve ensures that the gas pressure delivered to the patient's lungs does not exceed the selected PIP pressure for the patient. This is an inhalation phase of a cycle in the rhythm. When the healthcare professional removes his/her thumb or finger from the PEEP valve to open the valve during an exhalation phase of a cycle in the rhythm, gas flow from the gas source flows through the PEEP valve against the selected pressure of the PEEP valve and not to the patient and, simultaneously, during this phase the patient exhales against the selected pressure of the PEEP valve, with the exhaled gas flowing through the PEEP valve. The blow-off pressure relief valve actuates automatically in situations where the gas pressure being delivered to the infant's lungs via the flow control unit and the gas source exceeds a selected pressure for the blow-off pressure relief valve, which is higher than the PIP pressure. The selected pressure for the blow-off pressure relief valve is set to ensure safe operation of the resuscitator for the patient. This is a particularly important consideration for infants.

The flow control unit may include a swivel coupling for connecting unit to the gas source.

The flow control unit may include a swivel coupling for connecting the unit to the face mask or endotracheal tube.

The flow control unit may include a swivel coupling for connecting unit to the gas source and a swivel coupling for connecting the unit to the face mask or endotracheal tube.

The PIP valve may be adjustable manually to change the PIP pressure as required during operation of the resuscitator.

The PEEP valve may be adjustable manually to change the PEEP pressure as required during operation of the resuscitator.

The flow control unit may be any suitable structure.

The flow control unit may be a cross-shaped structure, which may be described as a hub structure, with the gas flow passage of the flow control unit having the following four branches extending from a central intersection:

(a) an inlet for gas flow from the gas source;

(b) an outlet for gas flow for inhalation by the patient and an inlet for exhalation from the infant;

(c) an assembly of the PIP valve and the blow-off pressure relief valve; and

(d) the PEEP valve.

The PIP valve and the blow-off pressure relief valve may be directly opposite the gas flow inlet.

The PEEP valve may be directly opposite the infant outlet/inlet.

The selection of the PIP, PEEP and blow-off pressures may be determined in any situation having regard to factors including but not limited to the age and health status of the infant, as determined by healthcare professionals responsible for the infant.

The assembly of the PIP valve and the blow-off pressure relief valve may be any suitable structure.

The assembly of the PIP valve and the blow-off pressure relief valve may be a mechanical structure.

The assembly of the PIP valve and the blow-off pressure relief valve may include a valve body that defines a gas flow passage and includes a wider diameter part with an external screw thread at a forward end and a narrower diameter part at a rearward end that are separated by a shoulder, an internally threaded valve cap that is mounted to the valve body via the external screw thread on the valve body and includes a least one opening, a closure member, for example in the form of a flat plate, that rests on the shoulder and closes the gas flow passage when in this position, and a spring that is located under compression within the valve passage and contacts the valve cap and the closure member and biases the closure member towards the shoulder to the closed position and requires a pressure that is greater than the PIP pressure to open the valve passage against the spring bias.

The valve cap may be manually rotated clockwise or anticlockwise and therefore move axially on the valve body, with the axial movement changing the compression force acting on the spring and therefore the biasing force against the closure member to keep the closure member in the closed position. In use, the biasing force determines the PIP pressure for the PIP valve. More particularly, the pressure within the gas flow passage of the unit has to exceed the pressure applied by the spring in order to force the closure member away from the shoulder and allow gas flow through the gas flow passage of the assembly.

The valve body is a friction fit within the cross-shaped structure that defines the branch of the unit that forms the assembly. In use, when there is a significant over-pressure in the gas flow passage of the unit that exceeds the selected blow-off pressure for the unit, the pressure overcomes the frictional engagement of the valve body and separates the valve body from the unit, thereby causing a significant and immediate reduction in pressure within the gas flow passage in the unit.

The invention also provides a coupling having an inlet end adapted to be connected to an upstream gas flow and an outlet end adapted to be connected to a downstream tubing for supplying gas to an end use device, such as a resuscitator, with the outlet end being formed to allow the connection of a smaller or a larger diameter tubing to the outlet end.

The inlet end may be a swivel coupling.

The outlet end may include a cylindrical mounting surface for the wider diameter tubing.

The outlet end also includes a fitting that includes a wider diameter section that fits with a friction fit over the cylindrical mounting surface and a narrower diameter section for an end of the wider diameter tubing.

The outlet end may include a nozzle part that defines a mounting surface for the smaller diameter tube.

The coupling may be a flow meter nipple.

### BRIEF DESCRIPTION OF THE DRAWINGS

The invention is described further by way of example only with reference to the accompanying drawings, of which:

FIG. 1 is perspective view of one embodiment of a resuscitator in accordance with the invention;

FIG. 2 is another view of the hand-held flow control unit and gas supply tubing of the resuscitator shown in FIG. 1 from a different orientation;

FIG. 3 is an assembly drawing of the flow control unit and gas supply tubing of the resuscitator shown in FIGS. 1 and 2;

FIG. 4 is an enlarged view of the pressure relief valve in the circled section of the flow control unit shown in FIG. 3;

FIG. 5 is cross-section along the line 5-5 of FIG. 4; and

FIG. 6 is an enlarged sketch of a perspective view of the gas supply flow meter nipple of the resuscitator shown in FIGS. 1 and 2.

### DETAILED DESCRIPTION OF A SPECIFIC EMBODIMENT

The embodiment of the resuscitator of the invention shown in the Figures is one of a number of possible embodiments of the invention.

The embodiment is described in the context of resuscitating an infant. However, it is noted that the invention is suitable for use more widely than for infants.

The resuscitator includes an oxygen/air blender 3 that is adapted to be connected to a source of air (not shown) and a source of oxygen (not shown), a flow meter 17 for controlling the flow rate of blended gas from the oxygen/air blender 3, a coupling in the form of a flow meter nipple 53, a hand-held flow control unit generally identified by the numeral 5, and a length of tubing 7 having fittings 13 at opposite ends that interconnects the gas flow meter nipple 53 and an inlet 51 of the flow control unit 5.

The tubing 7 may be any suitable diameter and length. Typically, the tubing 7 is 2-2.5 m long and 10 mm inner diameter. As is described further below, the flow meter nipple 53 can be used with a narrower and a larger diameter tubing 7.

The purpose of the resuscitator is provide controlled “breaths” of blended gas to the lungs of an infant or other patient by way of forced inhalation for the infant and a set resistance to exhalation of the infant.

The flow control unit 5 is formed to include the important operating controls for setting and adjusting PIP and PEEP, a pressure gauge 9 for measuring the pressure of gas flowing through the flow control unit 5 from the oxygen/air blender 3, and a pressure relief blow-off valve in a compact unit that can be located near the head of a patient and can be held and operated comfortably in one hand. In particular, the pressure relief blow-off valve operates automatically when the pressure exceeds a safe threshold.

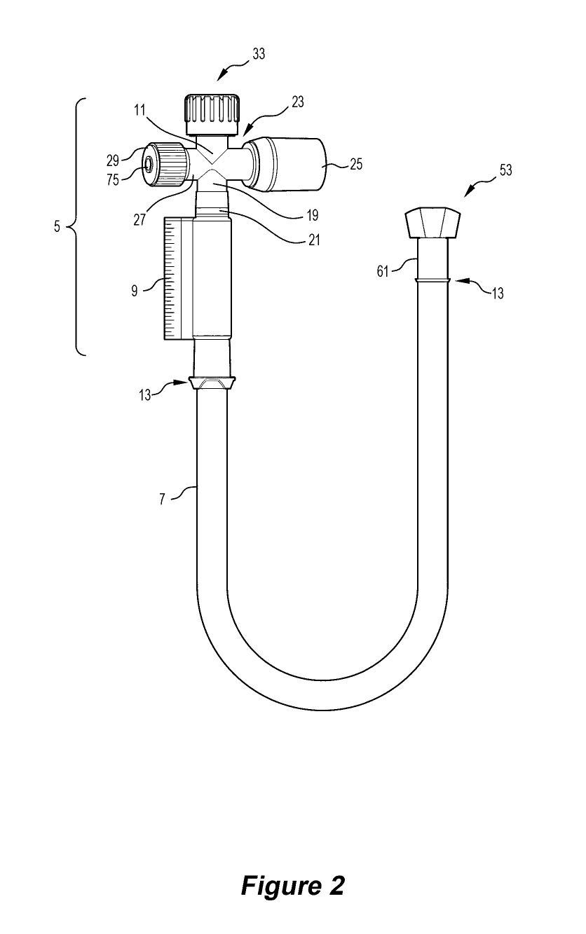

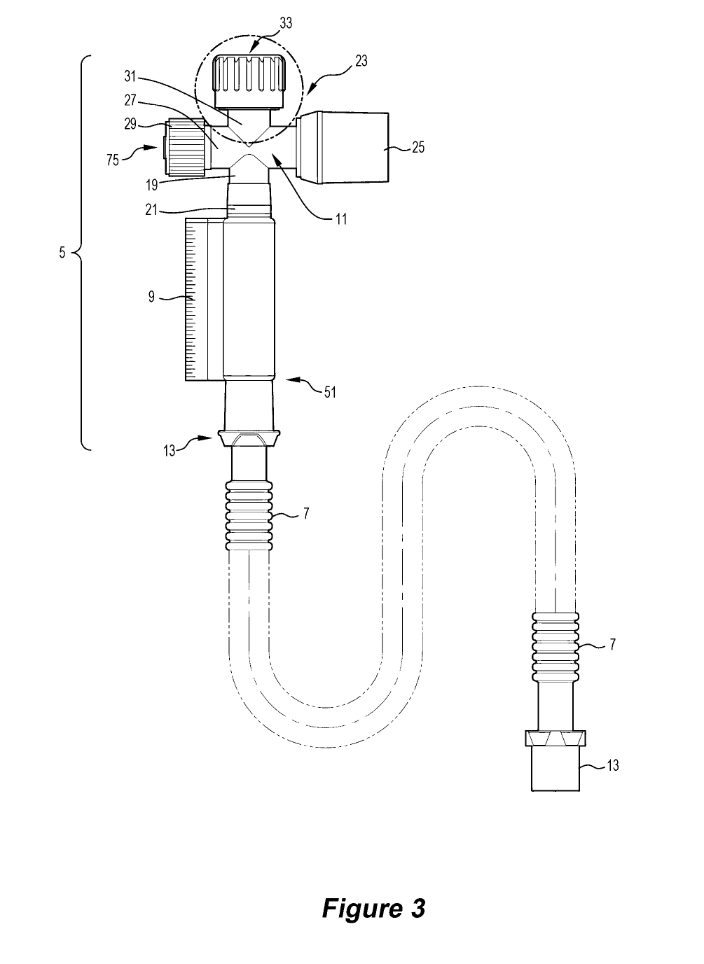

With reference particularly to FIGS. 2 and 3, the flow control unit 5 includes a cross-shaped hub structure 11, with includes a gas flow passage having four branches extending from a central intersection.

One branch is an inlet branch 19 for gas flow into the flow control unit 5 includes a pressure gauge 9 and a swivel coupling 21. The pressure gauge 9 is positioned to be conveniently in the line of sight of a healthcare professional using the resuscitator and is a convenient location to be viewed by the healthcare professional operating the resuscitator. The swivel coupling 21 may be any suitable coupling to allow swivelling movement of the flow control unit 5 with respect to the tubing 7.

Another branch 31 includes an assembly 33 of (a) a PIP valve and controller (“PIP valve”) to prevent gas flow to an infant above a selected PIP pressure, with the PIP valve 25 including a manually adjustable controller to change the PIP pressure and (b) a pressure relief blow-off valve for allowing pressure release from the flow control unit 5 when the pressure in the gas flow passage exceeds a selected pressure.

Another branch 27 includes a PEEP valve 29 for allowing gas flow through the valve above a selected PEEP pressure, which typically is lower, and more typically considerably lower, than the PIP pressure. The PEEP valve 29 includes a normally-open opening 75 that allows gas flow from the flow control unit 5. The PEEP valve 29 includes a manually adjustable flow controller to change the PEEP pressure. The purpose of the PEEP valve is to ensure that there is a positive pressure in the lungs of the infant after expiration. The PEEP valve 29 allows a healthcare professional to set a required PEEP pressure, as shown by the pressure gauge 9, to be retained within an infant's lungs after expiration. The pressure prevents the lungs sticking together, which is problematic for infants, particularly premature babies.

The fourth branch 23 is an outlet/inlet branch for connection to a face mask (not shown) over the nose and mouth of an infant or an endotracheal tube (not shown) of the infant for delivering gas to the infant and receiving exhaled gas from the infant. The branch 23 includes a swivel coupling 25. The swivel coupling 25 may be any suitable coupling to allow swivelling movement of the flow control unit 5 with respect to the face mask (not shown) or the endotracheal tube (not shown).

The double swivel valve arrangement, with the swivel couplings 21, 25, improves the functionality of the unit 5.

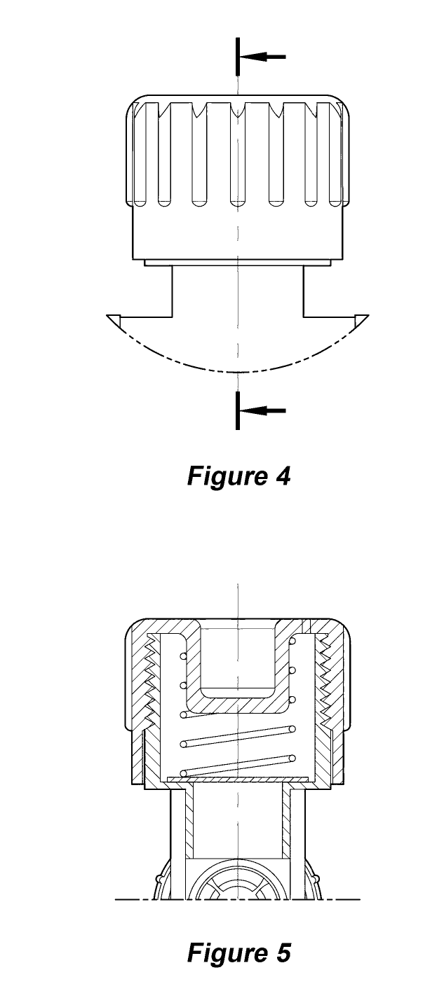

With reference to FIGS. 4 and 5, the assembly 33 of the PIP valve and the pressure relief valve includes a cylindrical valve body generally identified by the numeral 35 that defines a gas flow passage 37 and includes a wider diameter part 49 with an external screw thread at a forward end and a narrower diameter part 47 at a rearward end that are separated by a shoulder 43, an internally threaded valve cap 41 that is mounted to the valve body 5 via the external screw thread on the valve body 35 and includes a plurality of small openings 71 (only one of which is shown in FIG. 5) in a top wall of the valve cap 41 to allow gas flow from the gas flow passage 37, a member 39 in the form of a flat plate that rests on the shoulder 43, as shown in FIG. 5, and closes the gas flow passage 37 when in this position, and a spring 45 that is located under compression within the valve passage 37 and contacts the valve cap 41 and the closure member 39 and biases the closure member 39 towards the shoulder 43 to the closed position and requires a pressure that is greater than the PIP pressure to open the valve passage against the spring bias.

The valve cap 41 can be manually rotated clockwise or anticlockwise and therefore moved axially on the valve body 41. It can be appreciated that this axial movement changes the compression force acting on the spring 45 and therefore the biasing force against the closure member 39 to keep the closure member 39 in the closed position. In use, the biasing force determines the PIP pressure for the PIP valve. More particularly, the pressure within the gas flow passage of the unit 5 has to exceed the pressure applied by the spring 37 in order to force the closure member 39 away from the shoulder 43 and allow gas flow through the gas flow passage 37 of the assembly 33.

The narrower diameter part 47 of the valve body 41 is a friction fit within the cross-shaped structure that defines the branch that forms the assembly 33. When there is a significant over-pressure in the gas flow passage of the unit 5 that exceeds the selected blow-off pressure for the unit 5, the pressure overcomes the frictional engagement of the narrower diameter part 47 of the valve body 41 and separates the valve body 41 from the unit 5, thereby causing a significant reduction in pressure within the gas flow passage in the unit 5. It is noted that frictional engagement of the narrower diameter part 47 of the valve body 41 and the cross-shaped structure that defines the branch that forms the assembly 33 is not the only option for a structure that allows separation of the valve body 41 from the unit 5.

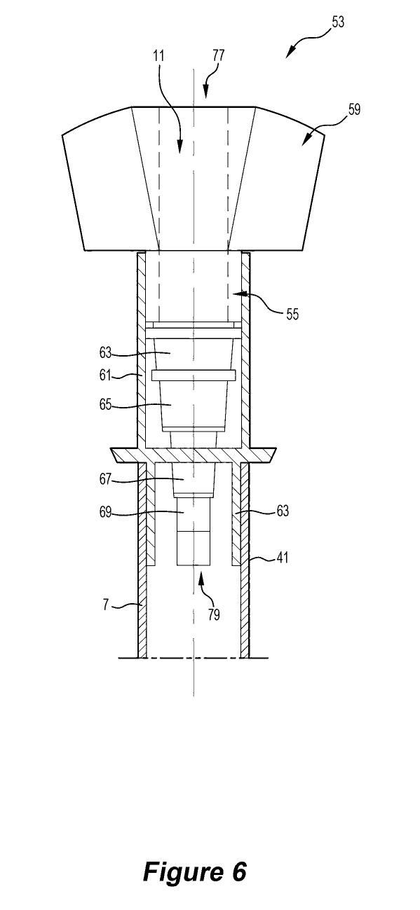

With reference to FIG. 6, the gas supply flow meter nipple 53 allows smaller or larger diameter tubing 7, typically 3-10 mm, to be connected between the flow meter 17 and the flow control unit 5. This feature improves the functionality of the resuscitator. The nipple 53 includes a central passageway 11 that has an inlet 77 and an outlet 79. The nipple 53 includes a cylindrical part 55 that defines a mounting surface for the tubing 7 and a series of successive smaller diameter mounting surfaces 63, 65, 67, 69 for successively narrower diameter tubing. The nozzle 53 also includes an optional adapter fitting that includes a wider diameter section 61 that fits with a friction fit over the cylindrical part 55 and a narrower diameter section 63 that defines a mounting surface for the tubing 7 and receives an end of the tubing 7. The nipple 53 also includes a swivel coupling 59 for connecting the nipple 53 to the flow meter 17.

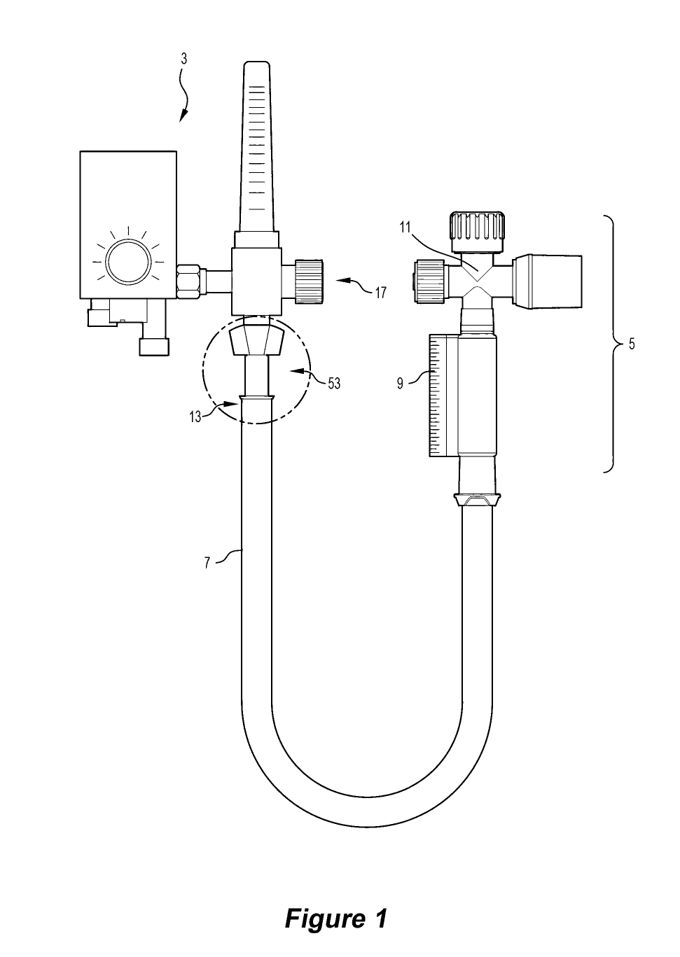

In use, as shown in FIG. 1, the hand held flow control unit 5 is connected directly to the oxygen/air blender 3 via the tubing 7, which typically is 2.0-2.5 m in length to provide operational flexibility and allow the healthcare professional to have better control of the tubing rather than being attached directly to a bench top resuscitator.

In a calibration stage, the flow control unit 5 is attached to a test lung (not shown). The healthcare professional then sets the gas flow rate from the gas source and the oxygen/air blender 3 to an initial flow rate (e.g. no more than 10 l/min). The healthcare professional then adjusts the controllers for the PIP and PEEP valves. The PIP valve is set first to a selected PIP pressure (e.g. between 10-40 cm H₂O). This setting regulates the controlled gas pressure delivered to the infant. The healthcare professional then adjusts the controller for the PEEP valve 29. The PEEP valve 29 is set to a selected PEEP pressure (e.g. between 3-8 cm H₂O). This setting controls the amount of gas held within the infant during exhalation. This is important to ensure that the infant's lungs do not stick together. The pressure gauge 9 is positioned conveniently to allow the pressures to be adjusted conveniently. The blow-off relief valve is also set to a selected pressure (e.g. 45 cm H₂O).

The flow control unit 5 is than coupled to a face mask or an endotracheal tube and healthcare professional commences the resuscitation of the infant.

Blended gas flows from the oxygen/air blender 3 and through the flow meter 17, the flow meter nipple 53, and the tubing 7 into the flow control unit 5 and from the flow control unit 5 via the PEEP valve 29 against the selected PEEP pressure and is exhausted from the flow control unit 5. The healthcare professional can view the pressure gauge 9 and see the gas pressure in the flow control unit as delivered via the oxygen/gas blender 3. The healthcare professional then connects the outlet branch 23 of the flow control unit 5 to a face mask (not shown) or an endotracheal tube (not shown) of a patient and selectively closes and opens the PEEP valve 29 to a required inhalation rhythm for the patient. When the PEEP valve 29 is closed, gas is forced to flow from the oxygen/gas blender 3 through the flow control unit 5 to the patient. When the PEEP valve 29 is open, gas flow from the oxygen/gas blender 3 flows through the PEEP valve 29 against the selected PEEP pressure of the PEEP valve 29 and not to the patient. Simultaneously, during this period the patient exhales against the selected PEEP pressure of the PEEP controller, with the exhaled gas flowing through the PEEP valve 29 and being exhausted from the flow control unit 5. The pressure relief valve actuates automatically in situations where the gas pressure in the flow control unit 5 exceeds a threshold pressure for the pressure relief valve. The threshold pressure is set to ensure safe operation of the resuscitator for the patient.

Many modifications may be made to the embodiment of the invention described above without departing from the spirit and scope of the invention.

By way of example, whilst the embodiment of the resuscitator shown in the Figures includes an oxygen/gas blender 3, it can readily be appreciated that the invention is not limited to this arrangement and other embodiments of the invention do not include an oxygen/gas blender 3.

By way of example, whilst the embodiment of the resuscitator shown in the Figures is described in the context of use for infants, it can readily be appreciated that the invention is not confined to use for infants.

1. A resuscitator that includes a flow control unit that, in use, can be positioned at the head end of a patient, such as an infant, and connected to a gas source and to a face mask or endotracheal tube for the patient for supplying air or oxygen or oxygen-enriched air (hereinafter referred to as “gas”) under pressure to the patient, with the flow control unit including: (a) a passage for gas from the gas source to flow through the unit to be delivered to the patient during inhalation and for gas to flow from the patient during exhalation, (b) a peak inspirational pressure valve and controller (“PIP valve”) for controlling the pressure of gas delivered to the lungs via the passage during inhalation so that the pressure does not exceed a selected PIP pressure, (c) a positive end-expiratory pressure valve and flow controller (“PEEP valve”) for allowing gas flow from the lungs via the passage and through the PEEP valve during exhalation when the pressure in the lungs is above a selected PEEP pressure to ensure that there is a minimum pressure (i.e. the selected PEEP pressure) of gas held in the lungs after exhalation, (d) a blow-off pressure relief valve that opens automatically if the gas pressure being delivered to the lungs via the passage exceeds a selected safe operation blow-off pressure that is higher than the selected PIP pressure; and (e) a pressure gauge for measuring the gas pressure in the passage.

2. The resuscitator defined in claim 1 wherein the flow control unit includes a swivel coupling for connecting unit to the gas source.

3. The resuscitator defined in claim 1 wherein the flow control unit includes a swivel coupling for connecting unit to the face mask or endotracheal tube.

4. The resuscitator defined in claim 1 wherein the flow control unit includes a swivel coupling for connecting unit to the gas source and a swivel coupling for connecting the unit to the face mask or endotracheal tube.

5. The resuscitator defined in claim 1 wherein the PIP valve is adjustable manually to change the PIP pressure as required during operation of the resuscitator.

6. The resuscitator defined in claim 1 wherein the PEEP valve is adjustable manually to change the PEEP pressure as required during operation of the resuscitator.

7. The resuscitator defined in claim 1 wherein the flow control unit is a cross-shaped structure, which may be described as a hub structure, with the gas flow passage of the flow control unit having the following four branches extending from a central intersection:
(a) an inlet for gas flow from the gas source;
(b) an outlet for gas flow for inhalation by the patient and an inlet for exhalation from the infant;
(c) an assembly of the PIP valve and the blow-off pressure relief valve; and
(d) the PEEP valve.

8. The resuscitator defined in claim 7 wherein the PIP valve and the blow-off pressure relief valve are directly opposite the gas flow inlet.

9. The resuscitator defined in claim 7 wherein the PEEP valve is directly opposite the infant outlet/inlet.

10. The resuscitator defined in claim 7 wherein the assembly of the PIP valve and the blow-off pressure relief valve includes a valve body that defines a gas flow passage and includes a wider diameter part with an external screw thread at a forward end and a narrower diameter part at a rearward end that are separated by a shoulder, an internally threaded valve cap that is mounted to the valve body via the external screw thread on the valve body and includes a least one opening, a closure member, for example in the form of a flat plate, that rests on the shoulder and closes the gas flow passage when in this position, and a spring that is located under compression within the valve passage and contacts the valve cap and the closure member and biases the closure member towards the shoulder to the closed position and requires a pressure that is greater than the PIP pressure to open the valve passage against the spring bias.

11. The resuscitator defined in claim 10 wherein the valve cap can be manually rotated clockwise or anticlockwise and therefore move axially on the valve body, with the axial movement changing the compression force acting on the spring and therefore the biasing force against the closure member to keep the closure member in the closed position.

12. The resuscitator defined in claim 11 wherein the valve body is a friction fit within the cross-shaped structure that defines the branch of the unit that forms the assembly whereby, in use, when there is a significant over-pressure in the gas flow passage of the unit that exceeds the selected blow-off pressure for the unit, the pressure overcomes the frictional engagement of the valve body and separates the valve body from the unit, thereby causing a significant and immediate reduction in pressure within the gas flow passage in the unit.

13. A coupling having an inlet end adapted to be connected to an upstream gas flow and an outlet end adapted to be connected to a downstream tubing for supplying gas to an end use device, such as a resuscitator, with the outlet end being formed to allow the connection of a smaller or a larger diameter tubing to the outlet end.

14. The coupling defined in claim 13 wherein the inlet end is a swivel coupling.

15. The coupling defined in claim 13 wherein the outlet end includes a cylindrical mounting surface for the wider diameter tubing.

16. The coupling defined in claim 15 wherein the outlet end also includes a fitting that includes a wider diameter section that fits with a friction fit over the cylindrical mounting surface and a narrower diameter section for an end of the wider diameter tubing.

17. The coupling defined in claim 15 wherein the outlet end includes a nozzle part that defines a mounting surface for the smaller diameter tube.

18. The coupling defined in claim 13 being in the form of a flow meter nipple.
